(19) United States
(12) Patent Application Publication
(10) Pub. No.: US 2022/0339122 A1
(43) Pub. Date: Oct. 27, 2022

(54) METHODS FOR THE TREATMENT OF DISEASES ASSOCIATED WITH DYSREGULATED ACTIVATION AND RECRUITMENT OF NEUTROPHILS

(71) Applicant: Chang Gung University, Taoyuan City (TW)

(72) Inventors: Tsong-Long HWANG, New Taipei City (TW); Ting-I KAO, Taipei City (TW); Tian-Shung WU, Tainan City (TW)

(73) Assignee: Chang Gung University, Taoyuan City (TW)

(21) Appl. No.: 17/241,064

(22) Filed: Apr. 27, 2021

(51) Int. Cl. A61K 31/085; A61P 11/16

(52) U.S. Cl. CPC A61K 31/085; A61P 11/16

(57) ABSTRACT

Disclosed herein is a method of treating diseases and/or disorders associated with the dysregulated activation and recruitment of neutrophils. The method includes administering to a subject in need thereof an effective amount of 3,3′-dihydroxy-2′,6′-bis(p-hydroxybenzyl)-5-methoxybibenzyl (or bletinib), a salt, a solvate or an ester thereof.

### STATEMENT REGARDING PRIOR DISCLOSURES BY THE INVENTOR OR A JOINT INVENTOR UNDER 37 C.F.R. 1.77(B)(6)

Part of the subject matter of the invention described in the present application was published by the inventors, Tsong-Long Hwang, Ting-I Kao, Tian-Shung Wu in an article titled “Bletinib ameliorates neutrophilic inflammation and lung injury by inhibiting Src family kinase phosphorylation and activity”. The article was published on Apr. 27, 2020 in the web site of British Journal of Pharmacology (See https://www.authorea.com/users/315805/articles/446056-bletinib-ameliorates-neutrophilic-inflammation-and-lung-injury-by-inhibiting-src-family-kinase-phosphorylation-and-activity). The publication was made by and/or originated from 3 members of the inventive entity of the present invention, and the entirety of this article is incorporated herein by reference. A copy of the article is provided in a concurrently filed Information Disclosure Statement pursuant to the guidance of 78 Fed. Reg. 11076 (Feb. 14, 2013).”

### BACKGROUND OF THE INVENTION

### 1. Field of the Invention

The present disclosure in general relates to novel uses of a natural bibenzyl, particularly, the use of 3,3′-dihydroxy-2′,6′-bis(p-hydroxybenzyl)-5-methoxybibenzyl (or bletinib) in treating diseases and/or disorders associated with dysregulated activation and recruitment of neutrophils, such as acute lung injury (ALI).

### 2. Description of Related Art

Neutrophils, the most abundant granulocytes in circulation, are responsible for eliminating pathogens through degranulation, enabling neutrophil elastase (NE) release, respiratory burst with superoxide production, and neutrophil extracellular trap (NET) formation. Thus, neutrophils are key effectors of both adaptive and innate immune systems. During inflammation, adhesion and migration are both crucial steps of neutrophil recruitment, which is regulated by the conformational change of macrophage-1 antigen (Mac-1; also known as aMβ2 and CD11b-CD18) on neutrophil surfaces. Dysregulated activation and recruitment of neutrophils can cause damage to host tissue through the release of excessive amounts of proteolytic enzymes, reactive oxygen species (ROS), and NETs, resulting in various morbidities, including autoimmune diseases (e.g., systemic lupus erythematosus, rheumatoid arthritis and psoriasis), infectious diseases (e.g., sepsis), inflammatory diseases (e.g., ARDS, chronic obstructive pulmonary disease and asthma), atherosclerosis, and other major diseases (e.g., cancers).

In traditional Chinese medicine, Bletilla tubers have been used to treat pulmonary, gastrointestinal, and dermatological inflammatory and haemorrhagic diseases for thousands of years. Bletinib (3,3′-dihydroxy-2′,6′-bis(p-hydroxybenzyl)-5-methoxybibenzyl) is a natural bibenzyl that was first extracted from Bletilla striata bulbs and has been reported to be antibacterial, antifungal, antiallergic, and antimitotic.

In this application, the inventors unexpectedly discovered that bletinib may regulate the inflammatory condition of activated human neutrophils, thus may serve as a candidate compound for the development of a medicament for treating diseases and/or disorders associated with the dysregulated activation and recruitment of neutrophils, such as ALI.

### SUMMARY OF THE INVENTION

The present disclosure provides novel use of a natural bibenzyl, i.e., 3,3′-dihydroxy-2′,6′-bis(p-hydroxybenzyl)-5-methoxybibenzyl (or bletinib), extracted from Bletilla formosana, which is found to suppress the dysregulated activated neutrophils, thus bletinib may serve as a candidate compound for the development of a medicament for treating diseases and/or disorders associated with the dysregulated activation and recruitment of neutrophils, such as ALI.

Accordingly, the first aspect of the present disclosure is directed to a method of treating a subject having an ALI. The method includes administering to the subject an effective amount of bletinib, a salt, a solvate or an ester thereof.

According to embodiments of the present disclosure, the ALI may be acute respiratory distress syndrome (ARDS).

Exemplary ARDS that may be treated by the present method includes, but is not limited to, transfusion-related lung injury, ventilator-induced lung injury, bacteria-induced lung injury, virus-induced lung injury, etc.

According to embodiments of the present disclosure, the bletinib is administered to the subject in the amount of 0.01 to 100 mg/Kg. Preferably, the bletinib is administered to the subject in the amount of 0.1 to 80 mg/Kg.

According to embodiments of the present disclosure, the subject suitable to be treated by the present method is a mammal; preferably, a human.

The details of one or more embodiments of this disclosure are set forth in the accompanying description below. Other features and advantages of the invention will be apparent from the detail descriptions, and from claims.

It is to be understood that both the foregoing general description and the following detailed description are by examples, and are intended to provide further explanation of the invention as claimed.

### BRIEF DESCRIPTION OF THE DRAWINGS

The accompanying drawings, which are incorporated in and constitute a part of the specification, illustrate various example systems, methods and other exemplified embodiments of various aspects of the invention. The present description will be better understood from the following detailed description read in light of the accompanying drawings, where,

FIG. 1 Bletinib suppressed superoxide anion production in stimulated human neutrophils. Neutrophils (6×10⁵ cells/mL) were incubated with 0.1% DMSO or 0.3-M bletinib for 5 min before activation with stimuli. Superoxide anion production was induced by (A) fMLF, (B) MMK-1, (C) NaF, or (D) PMA and then measured using ferricytochrome c reduction method. (E) Cytotoxicity was assessed using LDH release. (F) Superoxide scavenging capacity of bletinib was assessed in a xanthine/xanthine oxidase cell-free system. WST-1 reduction was measured through spectrophotometry at 450 nm, with 20 U/mL superoxide dismutase as the positive control. All data are displayed as box-and-whiskers plots, median (min-max; n=6). *p<0.05 compared with the DMSO+fMLF group.

FIG. 2 Bletinib reduced ROS production in fMLF-stimulated neutrophils. (A) Human neutrophils labelled with DHR123 were pretreated with 0.1% DMSO or 0.1-3 μM bletinib, activated with 0.1 μM fMLF, and then monitored through flow cytometry. The intracellular ROS levels and mean fluorescence intensity (MFI) of DHR123 in the neutrophils are presented as a box-and-whiskers plot, median (min-max; n=5). (B) Neutrophils were incubated with 0.1-3 μM bletinib or 0.1% DMSO for 5 min with or without 0.1 μM fMLF. The change in chemiluminescence was monitored in real time using an ELISA reader. The peak chemiluminescence is displayed as box-and-whiskers plots, median (min-max; n=5; right panel). *p<0.05 compared with the DMSO+fMLF group.

FIG. 3 Bletinib attenuated NE release in activated neutrophils. Neutrophils were treated with 0.1-10 μM bletinib or 0.1% DMSO for 5 min, and NE release was induced by (A) fMLF, (B) MMK-1, (C) NaF, or (D) LTB₄ along with the NE substrate and then evaluated spectrophotometrically. Data are expressed as box-and-whiskers plots, median (min-max; n=6). *p<0.05 compared with the DMSO+fMLF group.

FIG. 4 Bletinib reduced NET formation in PMA-stimulated neutrophils. Human neutrophils were pretreated with 0.1% DMSO or 1-10 μM bletinib for 10 min and then incubated with or without 10 nM PMA. (A) NET formation was quantified using Sytox green, a nucleic acid stain. (B) Neutrophils were stained with antibodies against NE (red) or MPO (green) and then analysed through confocal microscopy. DNA was detected using Hoechst 33342 (blue). (C) Scanning electron microscopy images of neutrophils. Representative images are shown. Data are expressed as box-and-whiskers plots, median (min-max; n=5). *p<0.05 compared with the DMSO+fMLF group.

FIGS. 5A to 5E Bletinib inhibited ERK and SFKs phosphorylation in fMLF-stimulated neutrophils. Neutrophils were preincubated with 0.1% DMSO or 10 μM Bnletinib and then stimulated with 0.1 μM fMLF. Immunoblots for phosphorylated and total (A) SFKs, (B) Akt (S473), (C) ERK, (D) JNK, and (E) p38 and the related quantifications are presented as box-and-whiskers plots, median (min−max; n=6). *p<0.05 compared with the DMSO+fMLF group.

FIGS. 6A to 6F Bletinib inhibited SFKs phosphorylation. Phosphorylation of SFKs, namely (A) Src, (B) Lyn, (C) Fgr, and (D) Hck, and downstream proteins, (E) Btk and (F) Vav, was independently determined through immunoblotting.

FIGS. 6G to 6J Bletinib inhibited enzymatic activity. The ADP-Glo kinase assay kit was used to evaluate the enzymatic activity of (G) Src, (H) Lyn, (I) Fgr, or (J) Hck (1.5 ng/mL), which was independently incubated with DMSO, 1-10 μM Bletinib, or 0.1-3 μM PP2, and then 125 μM ATP (substrate) was added to the reaction mixture for 60 min, enzyme activity was then determined. Data are expressed as box-and-whiskers plots, median (min-max; n=6). *p<0.05 compared with the DMSO+fMLF group.

FIG. 7 Bletinib inhibited adhesion and transmigration of fMLF-activated human neutrophils. Hoechst 33342-labelled neutrophils (10⁶ cells/mL) were treated with 0.1% DMSO or 1-10 μM bletinib for 5 min, followed by no stimulation or stimulation with 0.1 μM fMLF/1 μg/mL CB for another 5 min. Neutrophils were then incubated with bEnd.3 cells at 37° C. for 30 min. The neutrophils adherent on bEnd.3 cells were detected and numerated through fluorescent microscopy. (A) Quantification of the adherent neutrophils. (B) Human neutrophils were treated with DMSO or 1-10 μM bletinib for 5 min in the top of the chemotaxis chamber and then were treated with or without 0.1 μM fMLF for another 60 min. Migrated neutrophils were measured using a cell counter. Data are expressed as box-and-whiskers plots, median (min−max; n=5). *p<0.05 compared with the DMSO+fMLF group.

FIG. 8 Bletinib decreased CD11b (integrin α_{M}) and CD18 (integrin (β₂) expression in fMLF-activated neutrophils. Neutrophils were incubated with 0.1% DMSO or 1-10 μM bletinib for 5 min and then either not stimulated or stimulated with 0.1 μM fMLF/1 g/mL CB for another 5 min. The mean fluorescence intensity (MFI) of FITC-labelled antibodies against (A) CD11b and (B) CD18 was detected through flow cytometry. Data are illustrated with box-and-whiskers plots, median (min-max; n=6). *p<0.05 compared with DMSO+fMLF group.

FIG. 9 Bletinib mitigated LPS-induced ALI in mice. BALB/c mice (n=6 in each group) were treated with the vehicle (10% DMSO) or 25 mg/kg bletinib through intraperitoneal injection followed by intratracheal spraying of LPS for 5 h. Light microscopy images of (A) the exterior of the lungs and (B) H&E-stained, Ly6G-positive, MPO-positive, NE-positive, IL-1β-positive, 4-HNE-positive, occludin-positive, and p-Vav-positive lung sections.

FIG. 10 Bletinib reduced LPS-induced NET formation in mice. BALB/c mice (n=6 in each group) were treated with the vehicle (10% DMSO) or 25 mg/kg bletinib through intraperitoneal injection followed by intratracheal spraying of LPS for 5 h. (A) MPO activity and (B) total protein concentration in the lung tissues were measured 5 h after LPS spraying. (C) Immunofluorescent images of DAPI-positive, Ly6G-positive, and citH3-positive lung sections are presented. *p<0.05 compared with the vehicle+LPS group, #p<0.05 compared with the vehicle alone group.

FIG. 11 Bletinib reduced LPS-induced mortality in mice. BALB/c mice (n=6 in each group) were treated with the vehicle (10% DMSO) or 25 mg/kg bletinib through intraperitoneal injection followed by intraperitoneal injection of LPS. The survival rate was monitored for 5 days. Data are expressed as box-and-whiskers plots, median (min-max; n=6). *p<0.05 compared with the vehicle+LPS group, #p<0.05 compared with the vehicle alone group.

### DETAILED DESCRIPTION OF THE PREFERRED EMBODIMENTS

The detailed description provided below in connection with the appended drawings is intended as a description of the present disclosure and is not intended to represent the only forms in which the present disclosure may be constructed or utilized.

### 1. Definitions

The term “salt” refers to pharmaceutical acceptable salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “administered”, “administering” or “administration” are used interchangeably herein to refer a mode of delivery, including, without limitation, intravenously, intramuscularly, intraperitoneally, intraarterially, intracranially, or subcutaneously administering an agent (e.g., a compound or a composition) of the present invention. In some embodiments, the compound of the present disclosure or a salt, a solvate thereof is formulated into tablets for oral administration. In other embodiments, the compound of the present disclosure or a salt, a solvate thereof is formulated into powders for mixed with suitable carrier (e.g., buffer solution) before use, such as intravenous injection.

An “effective amount” of a compound described herein (either taken alone or in combination of another agent) refers to an amount sufficient to elicit the desired biological response, e.g., inhibiting the activation of inflammation or alleviating a target disease described herein or a symptom associated with the disease. As will be appreciated by those of ordinary skill in this art, the effective amount of a compound described herein may vary depending on such factors as the desired biological endpoint, the pharmacokinetics of the compound, the condition being treated, the mode of administration, and the age and health of a subject. In some examples, an effective amount can be a therapeutically effective amount, which refers to an amount of a therapeutic agent, alone or in combination with other therapies, sufficient to provide a therapeutic benefit in the treatment of a condition or to delay the onset or minimize one or more symptoms associated with the condition. The therapeutically effective amount refers to an amount that improves overall therapy, reduces or avoids symptoms, signs, or causes of the condition, and/or enhances the therapeutic efficacy of another therapeutic agent. In other examples, the effective amount can be a prophylactically effective amount. A prophylactically effective amount of a compound means an amount of a therapeutic agent, alone or in combination with other agents, which provides a prophylactic benefit in the prevention of the condition. For example, a prophylactically effective amount of a compound can be an amount sufficient to prevent or delay the onset of a condition, or one or more symptoms associated with the condition or prevent its recurrence. It may also be an amount that improves overall prophylaxis or enhances the prophylactic efficacy of another prophylactic agent.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The singular forms “a”, “and”, and “the” are used herein to include plural referents unless the context clearly dictates otherwise.

### 2. Use of the Compound of the Present Invention

The present disclosure lies in the unexpected discovery of a natural 3,3′-dihydroxy-2′,6′-bis(p-hydroxybenzyl)-5-methoxybibenzyl (or bletinib), extracted from Bletilla formosana possesses therapeutic effects toward dysregulated activated human neutrophils. Accordingly, bletinib may be used as a candidate compound for the development of medicaments suitable for treating diseases or disorders associated with dysregulated activation and recruitment of neutrophils, such as acute lung injury (ALI) and the like.

Accordingly, it is the first aspect of the present disclosure to provide a method of treating a subject suffering from an acute lung injury. The method comprises administering to the subject an effective amount of bletinib, a salt, a solvate or an ester thereof.

The compound of the present invention is available commercially or may be isolated by any methods known in the art, for example, the method described previously by Lin et al (J. Nat. Prod. 2016, 79, 1911-1921). Bioactivity analysis of the present compound indicates that it is a powerful inhibitory agent toward superoxide anion production, reactive oxygen species (ROS) production, and degranulation in stimulated human neutrophils. Furthermore, bletinib did not exhibit cytotoxicity, or superoxide scavenging activity in the xanthine/xanthine oxidase cell-free system. Findings of the present disclosure confirm that bletinib may serve as a candidate compound for the development of medicaments suitable for treating diseases associated with dysregulated activation and recruitment of neutrophils, such as ALI and the like.

Exemplary ALI that may be treated by the present method includes, but is not limited to, acute respiratory distress syndrome (ARDS), which may be resulted from transfusion-related lung injury, ventilator-induced lung injury, bacteria-induced lung injury, virus-induced lung injury, and etc.

According to embodiments of the present disclosure, the compound of the present disclosure, that is, 3,3′-dihydroxy-2′,6′-bis(p-hydroxybenzyl)-5-methoxybibenzyl (or bletinib), is administered to the subject in the amount of 0.01 to 100 mg/Kg, such as 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 mg/Kg; preferably, the 3,3′-dihydroxy-2′,6′-bis(p-hydroxybenzyl)-5-methoxybibenzyl (or bletinib) is administered to the subject in the amount of 0.1 to 80 mg/Kg, such as 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 mg/Kg. In one preferred embodiment, bletinib is administered to the subject in the amount of 2 mg/Kg. The effective amount of a compound may be administered in one or more doses for one or several days (depending on the mode of administration).

The present compound may also be formulated with suitable carriers or excipients for a suitable administration route, e.g., orally, parenterally, by inhalation spray, topically, rectally, nasally, buccally, vaginally or via an implanted reservoir. The term “parenteral” as includes subcutaneous, intracutaneous, intravenous, intramuscular, intraarticular, intraarterial, intrasynovial, intrasternal, intrathecal, intralesional and intracranial injection or infusion techniques.

A sterile injectable formulation, e.g., a sterile injectable aqueous or oleaginous suspension, can be formulated according to techniques known in the art using suitable dispersing or wetting agents (such as TWEEN® 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

A formulation suitable for oral administration can be any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the compound of the present disclosure can be suspended or dissolved in an oily phase combined with emulsifying or suspending agents. If desired, certain sweetening, flavoring, or coloring agents can be added. A nasal aerosol or inhalation formulation can be prepared according to techniques well-known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art. The compound of the present disclosure can also be administered in the form of suppositories for rectal administration.

Pharmaceutically acceptable carriers or excipients that may be included in a formulation comprising the compound of the present disclosure include inert diluents, solubilizing agents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the pharmaceutical composition.

An excipient present in an inventive formulation must be “pharmaceutically acceptable” in the sense that the excipient is compatible with the active ingredient of the pharmaceutical composition (and preferably, capable of stabilizing the pharmaceutical composition) and not deleterious to a subject to whom the pharmaceutical composition is administered. For example, solubilizing agents such as cyclodextrins, which may form specific, more soluble complexes with the compounds of the invention, can be utilized as pharmaceutically acceptable excipients for delivery of the compounds of the invention into the subject. Examples of other pharmaceutically acceptable excipients include colloidal silicon dioxide, magnesium stearate, cellulose, sodium lauryl sulfate, and D&C Yellow #10.

Also disclosed herein are kits (e.g., pharmaceutical packs) comprising the compound described herein, and a container (e.g., a vial, ampule, bottle, syringe, and/or dispenser package, or other suitable container). In some embodiments, the kits may include a second container comprising a pharmaceutically acceptable excipient for dilution or suspension of an inventive formulation. In some embodiments, the inventive formulation or compound provided in the first container and the second container are combined to form one unit dosage form.

In certain embodiments, a kit as described herein is for use in inhibiting the dysregulated activation and recruitment of neutrophils. In certain embodiments, a kit as described herein is for use in treating any of the target diseases as described herein (e.g., ALI) in a subject in need thereof. Any of the kits described herein can thus include instructions for administering the compound or pharmaceutical composition contained therein. A kit of the invention may also include information as required by a regulatory agency such as the FDA. In certain embodiments, the kit and instructions provide for treating a disease described herein. In certain embodiments, the kit and instructions provide for preventing a disease described herein. A kit of the invention may include one or more additional pharmaceutical agents described herein as a separate composition.

A “subject” to be treated by the method described herein can be a human subject (e.g., a pediatric subject such as an infant, a child, or an adolescent, or an adult subject such as a young adult, middle-aged adult, or senior adult), or a non-human animal, such as dogs, cats, cows, pigs, horses, sheep, goats, rodents (e.g., mice, rats), and non-human primates (e.g., cynomolgus monkeys, rhesus monkeys). The non-human mammal may be a transgenic animal or genetically engineered animal. In some examples, the subject is a human patient having a target disease as described herein (i.e., ARDS), suspected of having the disease, or is at risk for the disease. In some embodiments, the subject is a human or non-human mammal having, suspected of having a condition secondary to dysregulated activation and recruitment of neutrophils (e.g., ARDS).

It will be also appreciated that a compound or formulation, as described herein, can be used in combination with one or more additional agents (e.g., therapeutically and/or prophylactically active agents) in any of the methods described herein. The compound or formulation can be administered in combination with additional agents that improve their activity (e.g., activity (e.g., potency and/or efficacy) in treating a disease described herein in a subject in need thereof, in preventing a disease described herein in a subject in need thereof, in inhibiting the activation of neutrophile in a subject. It will also be appreciated that the therapy employed may achieve a desired effect for the same disorder, and/or it may achieve different effects.

The present invention will now be described more specifically with reference to the following embodiments, which are provided for the purpose of demonstration rather than limitation. While they are typically of those that might be used, other procedures, methodologies, or techniques known to those skilled in the art may alternatively be used.

### EXAMPLES

Materials and Methods

Preparation of Bletinib

Bletinib was extracted and purified in accordance with the process described previously (Lin et al., J. Nat. Prod. 2016 79, 1911-1921). Briefly, bletinib was extracted from the rhizomes of B. formosana with ethanol at 60° C. and purified through column chromatography. Bletinib (purity >98%) was dissolved in dimethyl sulfoxide (DMSO) for further experimentation. The control concentration of DMSO used in the cell experiments was 0.1%, which did not affect the parameters measured.

Animals

The animal care and experiment protocols were approved by the Institutional Animal Care and Use Committee of Chang Gung University, Taiwan. Moreover, the animal studies were reported in accordance with the ARRIVE (Animal Research: Reporting of In Vivo Experiments) guidelines. All the experimental procedures complied with The Guide for the Care and Use of Laboratory Animals (National Research Council Committee for the Update of the Guide for the Care and Use of Laboratory, 2011). Specified pathogen-free (SPF) 8-week-old male BALB/c mice (body weight: 20±1 g) were purchased from BioLASCO (Taiwan). Five mice shared a ventilated cage with standard bedding, and were given ad libitum access to water and standard laboratory chow. All mice were all kept in an SPF animal facility under a 12-12-h light-dark cycle. Mice were acclimatized for at least 1 week before use in the experiments.

LPS-Induced ALI and Mortality Model

A total of 24 male BALB/c mice were randomly divided into four groups (6 mice/group): vehicle alone, bletinib alone, LPS control, and bletinib treatment (bletinib+LPS). The mice were starved overnight and then intraperitoneally injected with 50 μL of bletinib (25 mg/kg) or 50 μL of vehicle (10% DMSO). ALI was induced through intratracheal spraying of 50 μL of LPS (from Escherichia coli O111:B4; 2 mg/kg) or 50 μL of 0.9% saline (in vehicle and bletinib alone group) under general anaesthesia with xylazine (6 mg/kg) and Zoletil 50 (30 mg/kg). Five hours later, mouse lungs were harvested after sacrifice and were frozen for myeloperoxidase (MPO) activity assay; they were also fixed with 10% formalin for histological sectioning and immunofluorescence staining.

For the LPS-induced mortality model, mice were intraperitoneally injected with a single 50-μL dose of LPS (from Escherichia coli O111:B4; 5 mg/kg) or 0.9% saline (vehicle alone group). The mice were monitored for 5 days to determine the survival rate.

Histological Sectioning and Immunofluorescence Staining

The harvested lung tissues were washed with phosphate-buffered saline (PBS) and fixed with 10% formalin for 24 h. The samples were subsequently dehydrated, embedded with paraffin, sliced into 3-μm-thick sections with a microtome, and placed on glass slides. These sections were stained using haematoxylin and eosin (H&E) and corresponding antibodies. Then, images were acquired through light microscopy.

For immunofluorescence staining, tissue sections were incubated with antibodies against H3 (citH3; citrulline R2+R8+R17) and Ly6G at dilutions of 1:800 and 1:200, respectively. Anti-IgG secondary antibodies labelled with a fluorescent dye (Alexa Fluor 488 for citH3 or Alexa Fluor 568 for Ly6G) were used at dilutions of 1:1000 and 1:500, respectively. Immunofluorescence images were acquired through confocal microscopy (LSM 510 Meta, Zeiss).

Analysis of Myeloperoxidase (MPO) activity

The mouse lung tissues were suspended in a 0.5% hexadecyltrimethylammonium bromide buffer (pH 6.0) and then homogenised through sonication. To evaluate MPO activity, the MPO substrate buffer (containing PBS, 0.0005% hydrogen peroxide, and 0.2 mg/mL 0-dianisidine hydrochloride) was added to the homogenised tissue, and the light absorbance at 460 nm was detected by spectrophotometry, after which, MPO activity was calculated with reference to the standard curve of human MPO activity.

Human Neutrophil Isolation

The study was conducted with the approval of the Institutional Review Board of Chang Gung Memorial Hospital (IRB No. 201601111A3) in accordance with the Declaration of Helsinki. After written informed consent was obtained, whole blood samples were drawn from healthy individuals aged 20-30 years who had not taken any medication within the previous 2 weeks. Neutrophils were then isolated using the standard procedures for Ficoll-Hypaque gradient centrifugation, dextran sedimentation, and hypotonic lysis of erythrocytes. The isolated neutrophils were then suspended in Ca²⁺-free HBSS (pH 7.4) and stored at 4° C. until use.

Measurement of Extracellular Superoxide Anion Production

Extracellular superoxide anion production in activated neutrophils was assessed through the reduction of ferricytochrome c. After incubating them with Ca²⁺ (1 mM) and ferricytochrome c (0.5 mg/mL) at 37° C., the isolated human neutrophils (6×10⁵ cells/mL) were then incubated with 0.1% DMSO or 0.3-10 μM bletinib for 5 min. The cells were pretreated with cytochalasin B (CB, 1 or 2 μg/mL) for 3 min and stimulated with fMLF, MMK-1, or sodium fluoride (NaF), or directly activated with phorbol-12-myristate-13-acetate (PMA). The change in absorbance at 550 nm was detected continuously using a spectrophotometer (U-3010, Hitachi, Tokyo, Japan), and superoxide anion levels were calculated using a method described previously (Hwang et al., 2003 Mol. Pharmacol. 64(6), 1419-1427).

Measurement of Intracellular Superoxide Anion Production

Human neutrophils (2.5×10⁶ cells/mL) were labelled using 2 μM dihydrorhodamine 123 (DHR123) at 37° C. for 10 min and then incubated with DMSO or bletinib for 5 min and then stimulated with 0.1 μM fMLF/0.5 μg/mL CB for 15 min. The fluorescence intensity was detected through flow cytometry to evaluate intracellular superoxide anion production of human neutrophils.

Analysis of Total ROS Production

Human neutrophils (2×10⁶ cells/mL) were preincubated with 6 U mL⁻¹ horseradish peroxidase (HRP) and 37.5 μM luminol in a 96-well plate at 37° C. for 5 min. Cells were incubated with DMSO or bletinib for 5 min, followed by 0.1 μM fMLF stimulation. Chemiluminescence was then detected and analysed in real time on a 96-well chemiluminometer (Tecan Infinite F200 Pro; Mannedorf, Switzerland).

Analysis of NE Release

Human neutrophils (6×10⁵ cells/mL) were incubated with DMSO or bletinib after treatment with 1 mM CaCl₂ and 100 μM NE substrate (Methoxysuccinyl-Ala-Ala-Pro-Val-p-nitroanilide) at 37° C. for 5 min. Cells were stimulated with fMLF/0.5 μg mL⁻¹ CB, leukotriene B4 (LTB4)/2 μg mL⁻¹ CB, NaF/2 μg mL⁻¹ CB, or MMK-1/0.5 μg mL-1 CB for 10 min before determination of NE release by measuring the change of absorbance at 405 nm in a spectrophotometer.

Analysis of Neutrophil Extracellular Trap (NET) Formation

Quantification of Extracellular DNA

Human neutrophils (10⁶ cells/mL) resuspended in HBSS with 2.5 μM Sytox green were incubated with DMSO or bletinib for 10 min and stimulated using 10 nM PMA or 10 μg/mL LPS for 3 h. The fluorescence intensity was quantified on a Tecan Infinite 200 reader at 485-535 nm.

NET Photography

Neutrophils (3×10⁵ cells/mL) were incubated with DMSO or bletinib for 10 min before being activated with 10 nM PMA for 2 h. Neutrophils were fixed with 4% paraformaldehyde and treated with 5% goat serum blocking buffer for 1 h and then treated with g/mL anti-MPO (Abcam) and 5 μg/mL anti-NE (Merck Millipore) antibodies for 1 h. These cells were then treated with the Alexa 488 or 568-labelled secondary goat anti-rabbit antibody for another 1 h. Thereafter, the cells were washed with PBS and treated with 1 ng/mL Hoechst 33342 and ProLong Gold antifade reagent (Invitrogen, CA, USA). Immunofluorescence microscopy and scanning electron microscopy were both used to observe the NET formation of activated neutrophils.

Evaluation of Neutrophil Adhesion

Human neutrophils (10⁶ cells/mL) were labelled with Hoechst 33342 and then incubated with DMSO or Bletinib for 5 min. After centrifugation, the cells were resuspended and activated with 0.1 μM fMLF/1 μg/mL CB for 10 min before incubation with bEnd.3 cells at 37° C. for 30 min. After they were washed with HBSS, the cells were fixed with 4% paraformaldehyde and the neutrophils that adhered to the bEnd.3 cells were detected and quantified on a motorised inverted microscope (Olympus, Japan).

Analysis of Neutrophil Migration

A microchemotaxis chamber with 3-μm filters (Millipore) was used to evaluate chemotactic migration of neutrophils. Neutrophils (5×10⁶ cells/mL) treated with bletinib or DMSO for 5 min were placed in the top chamber, and 0.1 μM fMLF was added into the bottom chamber. The number of neutrophils that migrated from the top to the bottom chamber after incubation at 5% CO₂ for 1 h was counted on a MoxiZ automatic cell counter (ORFLO).

Evaluation of surface CD18 and CD11b expression

Neutrophils (5×10⁵ cells/mL) were incubated with bletinib or DMSO for 5 min and then activated through incubation with 0.1 μM fMLF/1 μg/mL CB for 5 min. After centrifugation at 200×g for 8 min at 4° C., the cells were resuspended in 5% bovine serum albumin with FITC-conjugated antibodies against CD18 or CD11b on ice in the dark for 15 min. The fluorescent intensity was then analysed through fluorescence-activated cell sorting.

Immunoblotting of Neutrophil Lysates

Neutrophils incubated with bletinib or DMSO at 37° C. for 5 min were stimulated with 0.1 μM fMLF for 30 s. The proteins were separated from neutrophil lysates through electrophoresis (12% sodium dodecyl sulphate-polyacrylamide gel electrophoresis) and then transferred to nitrocellulose membranes. The target proteins were identified through immunoblotting with specific antibodies against p38, p-p38, Akt, p-Akt S473, ERK, p-ERK, INK, p-JNK, Src, p-SFKs Y416, p-Src Y416, Lyn, p-Lyn (Y396), Fgr, p-Fgr (Y412), Hck, p-Hck (Y410), Btk, p-Btk Y223, Vav, and p-Vav (Y174) as well as with HRP-conjugated secondary anti-rabbit antibody (Cell Signaling Technology). The signal intensity was detected and quantified using the UVP BioSpectrum Imaging System (Analytik Jena, USA).

Assessment of Enzymatic Activity of SFKs

The kinase activity of SFKs was assessed using an ADP-Glo kinase assay kit (Promega, Fitchburg, USA) according to manufacturer instructions. In brief, the kinase reaction was initiated by adding SFKs (Src, Lyn, Fgr, and Hck), their substrate 125 μM ATP, and 1-10 μM bletinib or 0.1-3 μM PP2 into the reaction buffer for 1 h. The ADP-Glo reagent was used to end the kinase reaction and remove the remnant ATP; next, the kinase, which converted ADP to ATP, detection reagent was added and incubated for 30 min. Luciferin/luciferase luminescence was determined on an Infinite 200 Pro (Tecan, Switzerland).

Statistical Data and Analysis

All data are presented as box-and-whiskers plots (median, min-max). One-way ANOVA and Dunnett's multiple comparison tests were employed for all experiments. The survival rate of the mice was analysed using the log-rank (Mantel-Cox) test. All statistical calculations were performed using GraphPad Prism software (GraphPad Software, San Diego, Calif., USA). Differences with p values <0.05 were considered significant statistically.

### Example 1 In Vitro Characterization of Bletinib

1.1 Bletinib Ameliorated Superoxide Anion Production and ROS Production in Stimulated Neutrophils

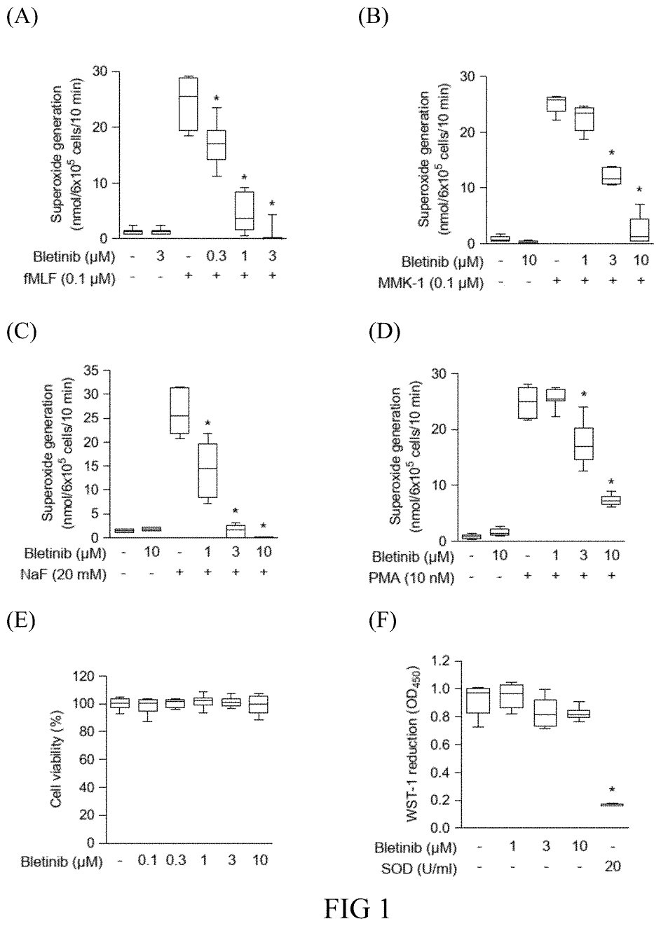

In this example, the modulatory effect of bletinib on inflammatory responses was investigated via monitoring the production of superoxide in human neutrophils stimulated with various chemoattractants, including formyl-L-methionyl-L-leucyl-L-phenylalanine (fMILF), NaF (a G protein activator), MMK-1 (a selective formyl peptide receptor 2 (FPR2) agonist), and phorbol 12-myristate 13-acetate (PMA). Results are illustrated in FIG. 1.

It was found that bletinib ameliorated superoxide anion produced by fMLF-activated human neutrophils in a dose-dependent manner (IC₅₀=0.62 0.15 μM; FIG. 1, (A)). Similarly, bletinib attenuated superoxide anion release from neutrophils stimulated by other chemoattractants, including MMK-1, sodium fluoride (NaF), and PMA (FIGS. 1, (B), (C) and (D)). Furthermore, bletinib did not exhibit cytotoxicity (FIG. 1, (F)) or ROS scavenging activity in the cell-free xanthine and xanthine oxidase system (FIG. 1, (G)).

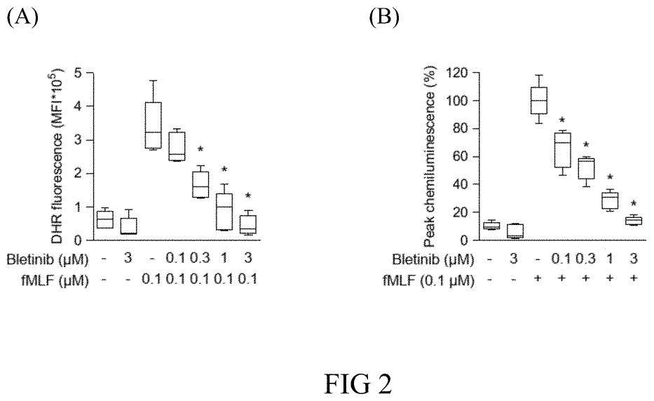

Flow cytometry and chemiluminescence assay were also performed to determine if bletinib affected ROS production in stimulated neutrophils. The quantified results of flow cytometry and luminol-amplified chemiluminescence assay revealed that bletinib significantly suppressed intracellular ROS production in fMLF-activated neutrophils in a dose-dependent manner (FIGS. 2, (A) and (B)).

1.2 Bletinib Inhibited Degranulation of Activated Human Neutrophils

In this example, the effect of bletinib on degranulation was investigated via measuring the release of neutrophil elastase (NE) in activated neutrophils, as degranulation is an important function of neutrophils during inflammation.

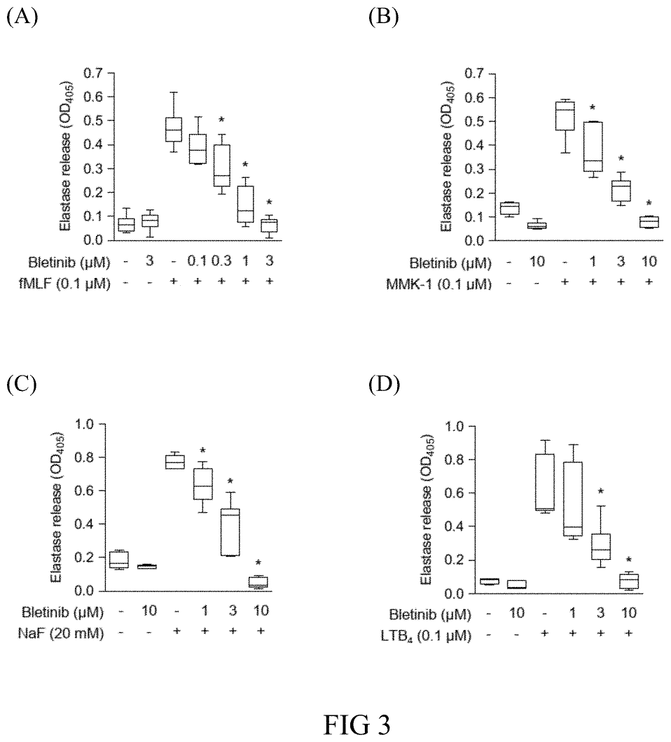

It was found that bletinib inhibited the release of NE in fMLF-stimulated human neutrophils (IC₅₀=0.53±0.07 μM) but not in the quiescent neutrophils (FIG. 3, (A)). In addition, bletinib downregulated NE release from neutrophils respectively stimulated with MMK-1, NaF, and LTB₄ in a concentration-dependent manner (FIGS. 3, (B), (C), and (D)).

1.3 Bletinib Attenuated Neutrophil Extracellular Trap (NET) Formation

NET, mainly composed of granular proteins, proteases, and chromatin filaments coated with histones, is crucial in inflammatory diseases and autoimmune disorders. To investigate the effects of bletinib on NET formation, neutrophils were stained with Sytox green after activation with PMA (10 nM) and LPS (10 μg/mL).

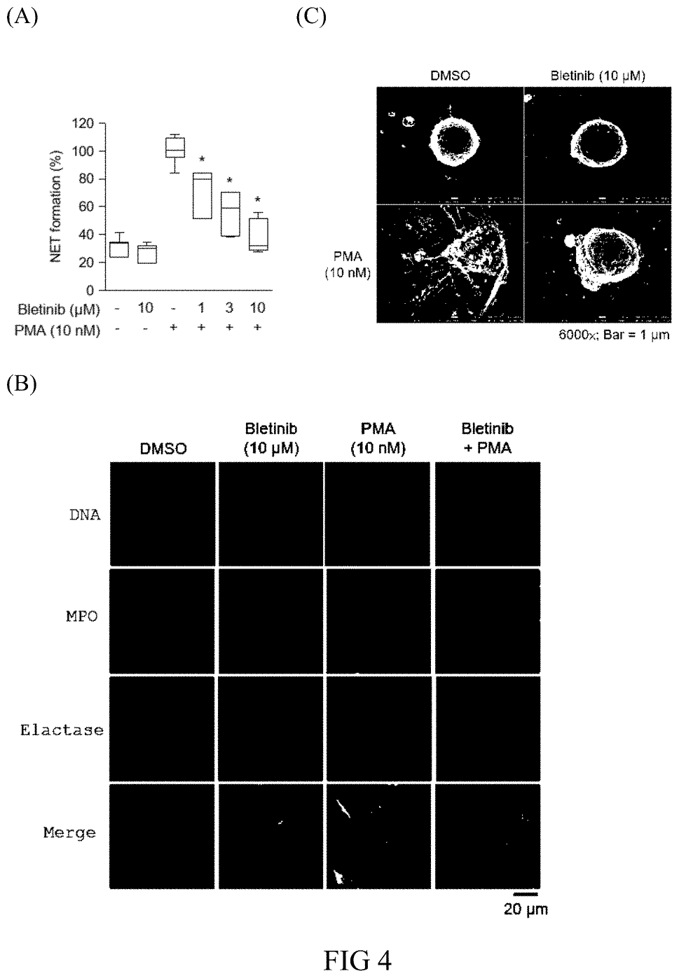

Fluorescent spectrometry assay results demonstrated that NET formation, induced by PMA, was significantly mitigated by bletinib (FIG. 4, (A)). Further, immunofluorescent staining demonstrated the presence of neutrophils co-stained with Hoechst 33342 and antibodies against MPO and NE in the NETs (FIG. 4, (B)). Images of immunofluorescent staining and scanning electron microscopy showed that bletinib effectively inhibited NET formation (FIGS. 4, (B) and (C)).

1.4 Bletinib Suppressed ERK and Src Family Kinase (SFK) Phosphorylation in fMLF-Activated Neutrophils

It is known that SFKs and the MAPK/ERK pathway play critical roles in the degranulation, respiratory burst, NET formation, and migration of neutrophils. Therefore, the effects of bletinib on the phosphorylation of SFKs, Akt, ERK, INK, and p38 in activated neutrophils were investigated.

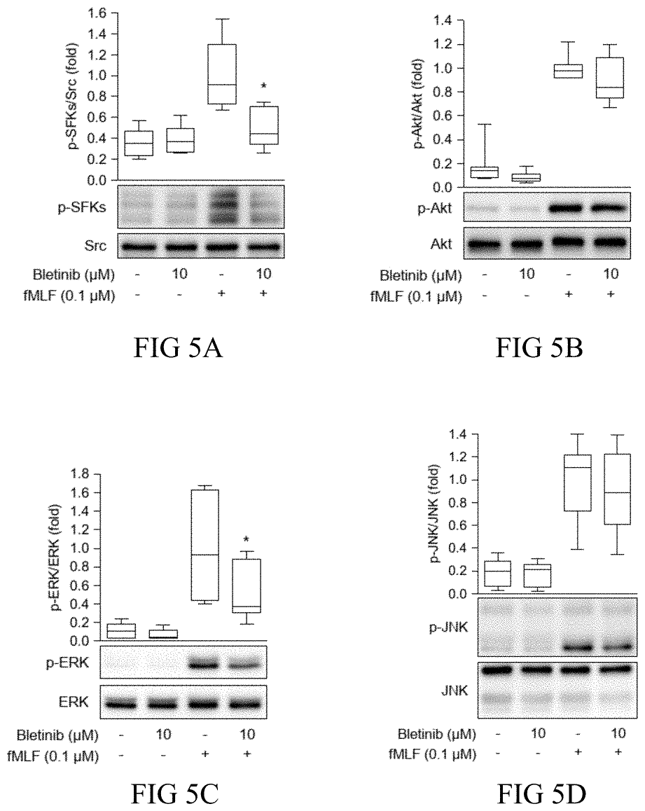

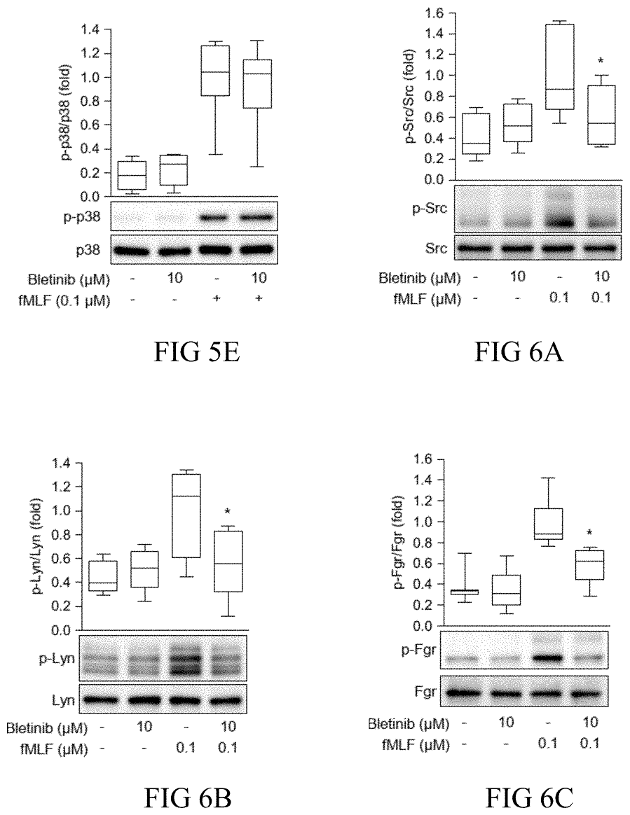

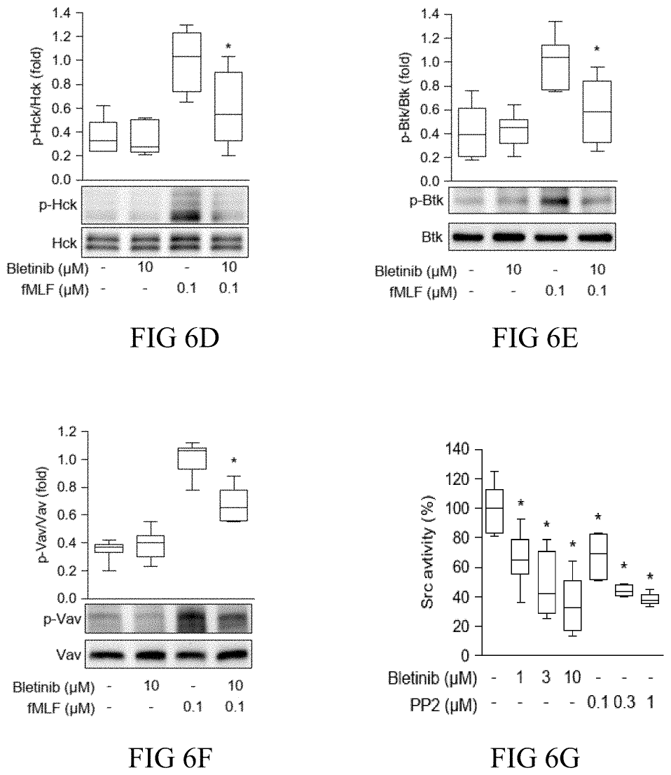

The immunoblotting results demonstrated that the phosphorylation of SFKs, Akt (S473), ERK, INK, p38, Src (Y416), Lyn (Y396), Fgr (Y412), Hck (Y410), Btk (Y223), and Vav (Y174) was enhanced in fMLF-stimulated neutrophils; however, bletinib significantly inhibited the phosphorylation of ERK, SFKs, Src (Y416), Lyn (Y396), Fgr (Y412), Hck (Y410), Btk (Y223), and Vav (Y174) but not that of Akt, JNK, and p38 (FIGS. 5, 6A to 6F).

1.5 Bletinib Inhibited SFK Activity

SFKs are nonreceptor tyrosine kinases present in neutrophils, with predominant expression of Src, Fgr, Hck, and Lyn. SFKs are responsible for the generation of the inflammatory environment in vivo. The cell-free ADP-Glo kinase assay confirmed that both bletinib and PP2, a selective inhibitor of SFKs, inhibited the kinase activity of Src, Fgr, Hck, and Lyn in a concentration-dependent manner (FIGS. 6G to 6J).

1.6 Bletinib Reduced Adhesion and Transmigration of Activated Human Neutrophils

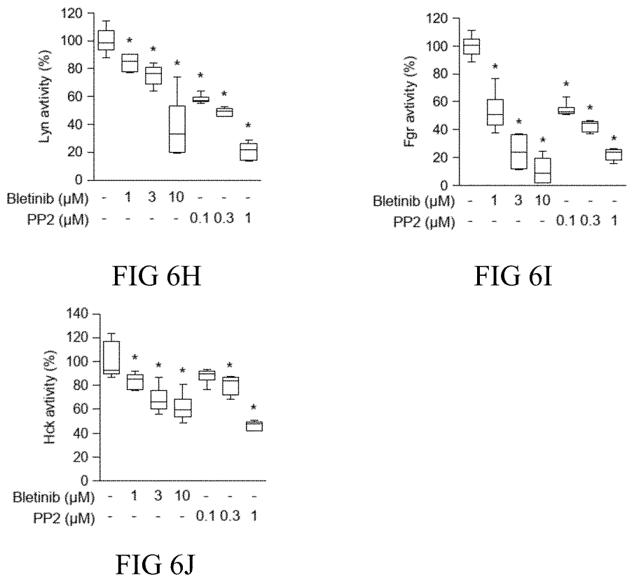

Adhesion and transmigration are both crucial steps in the neutrophil recruitment cascade during inflammation. In this example, to examine whether bletinib would interfere the adhesion and transmigration process, human neutrophils (10⁶ cells/mL) were first labeled with Hoechst 33342, then pre-treated with bletinib (1-10 μM) and followed by stimulation with fMLF, then incubated with bEnd.3 cells in 37° C. for 30 min. Neutrophils adhered on bEnd.3 cells were detected and counted using fluorescence microscopy, and the quantified results demonstrated that bletinib inhibited the adhesion function of fMLF-activated neutrophils (FIG. 7, (A)). Moreover, we used a chemotaxis chamber and a cell counter to enumerate the number of migrating neutrophils and found that bletinib significantly reduced fMLF-induced transwell migration of neutrophils (FIG. 7, (B)). Furthermore, IL-8 served as a chemotactic factor for the attraction of neutrophils. IL-8-induced neutrophil migration was also inhibited by bletinib (data not shown).

1.7 Bletinib Reduced Mac-1 Expression in Activated Neutrophils

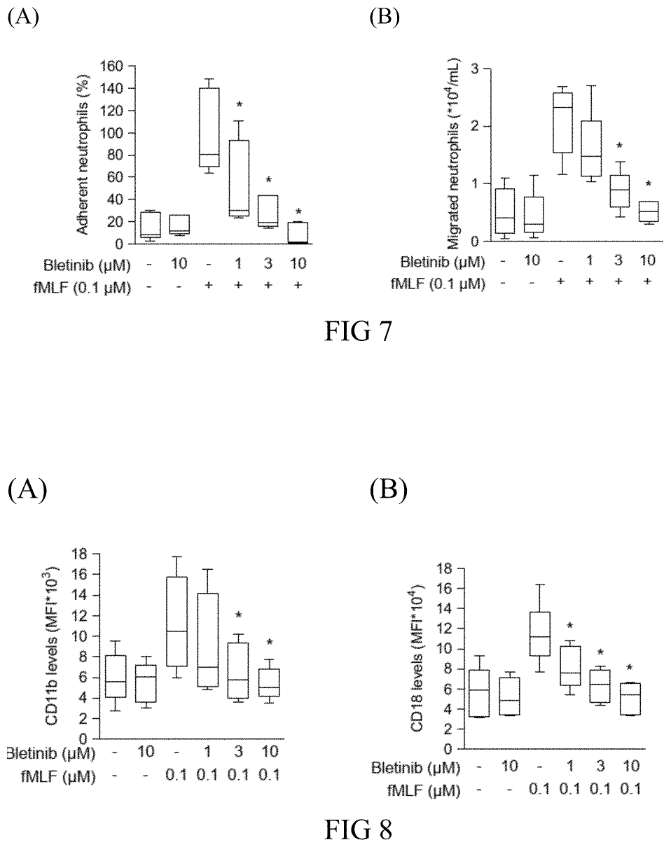

Mac-1 is a complement receptor composed of CD11b (integrin α_{M}) and CD18 (integrin β₂) and facilitates leukocyte recruitment during inflammation. Accordingly, the surface expression of CD11b and CD18 on neutrophiles was determined by flow cytometry and the results indicated that bletinib significantly attenuated the expression of both CD11b and CD18 in fMLF-stimulated human neutrophils (FIG. 8). Furthermore, the IL-8-induced CD11b expression was restrained by bletinib in human neutrophils (data not shown).

### Example 2 Bletinib Alleviated Lipopolysaccharide (LPS)-Induced Acute Lung Injury (ALI) and Mortality in Mice

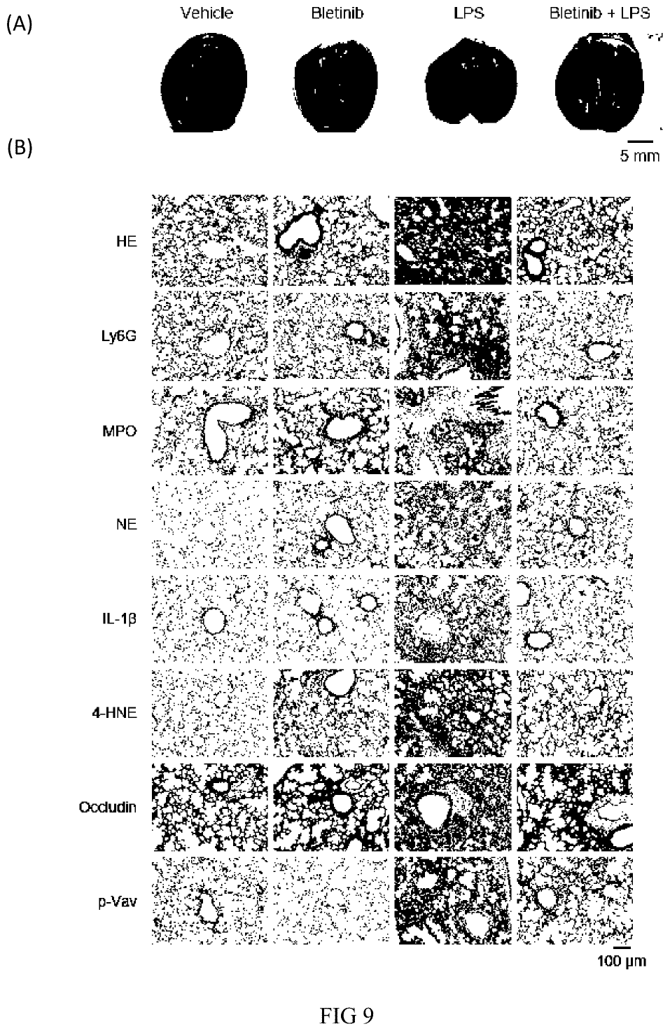

In this example, the anti-inflammatory effect of bletinib in vivo was investigated. To this purpose, BALB/c mice were treated with bletinib (25 mg/kg) or DMSO administered through intraperitoneal injection followed by intratracheal spraying of LPS for 5 h. The exterior photos and HE-stained histopathological features of lungs revealed that LPS induced haemorrhagic and erythematous conditions, interalveolar septal thickening, and pulmonary interstitial oedema formation (FIG. 9). Furthermore, infiltration of inflammatory MPO- and Ly6G-positive cells (specific markers of neutrophils), protease release (NE), cytokine production (IL-10), oxidative stress-induced lipid peroxidation (4-HNE), and vascular permeability (occludin) were observed after LPS administration. Distortion of the pulmonary architecture was significantly suppressed in the bletinib treatment group. In addition, LPS-activated phosphorylation of Vav (p-Vav) was also attenuated by bletinib (FIG. 9).

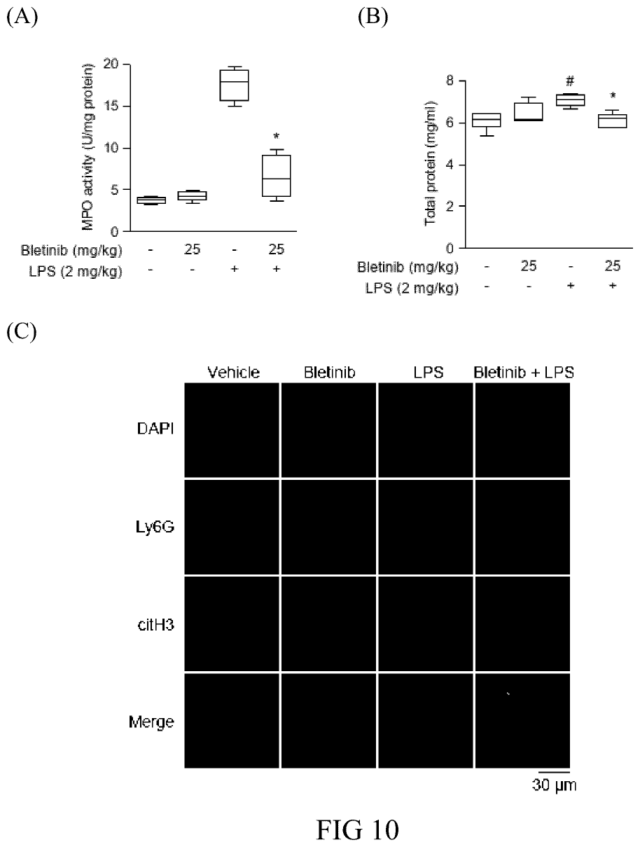

The MPO activity and total protein levels in the mouse lung tissues were determined to represent the severity of pulmonary oedema, which was increased in the control group but significantly ameliorated in the bletinib treated group (FIGS. 10, (A) and (B)). Furthermore, LPS-induced NET formation (Ly6G⁺citH3⁺ cell accumulation) was considerably reduced after bletinib treatment (FIG. 10, (C)).

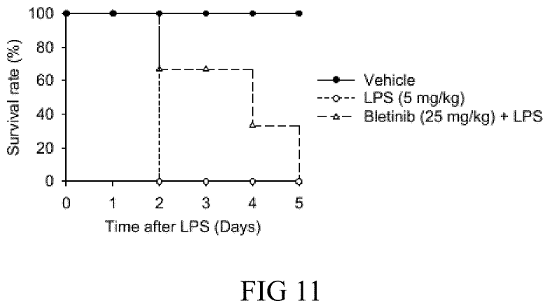

The therapeutic ability of bletinib to increase survival was further observed in LPS-primed mice. BALB/c mice were injected with LPS (5 mg/Kg) and their survival rate was monitored for 5 days. All mice in the control group died within 2 days, whereas the survival time of each bletinib-treated mice (25 mg/Kg) was significantly prolonged (log-rank test, p=0.0085; FIG. 11).

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the scope of the present disclosure.

What is claimed is:

1. A method of treating a subject having an acute lung injury (ALI) comprising administering to the subject an effective amount of bletinib.

2. The method of claim 1, wherein the bletinib is administered to the subject in the amount of 0.01 to 100 mg/Kg/day.

3. The method of claim 1, wherein the ALI is acute respiratory distress syndrome (ARDS).

4. The method of claim 3, wherein the ARDS is transfusion-related lung injury, ventilator-induced lung injury, bacteria-induced lung injury, or virus-induced lung injury.

5. The method of claim 1, wherein the subject is a mammal.

6. The method of claim 5, wherein the subject is a human.
